(19) United States
(12) Patent Application Publication
(10) Pub. No.: US 2004/0146871 A1
(43) Pub. Date: Jul. 29, 2004

(54) Method and high throughput adaptation thereof for RNA, DNA or protein isolation from animal tissues

(76) Inventors: Peter R. Hoyt, Oak Ridge, TN (US); Mitchel J. Doktycz, Knoxville, TN (US)

Correspondence Address: QUARLES & BRADY LLP, 411 E. WISCONSIN AVENUE, SUITE 2040, MILWAUKEE, WI, 53202-4497, US

(21) Appl. No.: 10/354,858

(22) Filed: Jan. 29, 2003

(51) Int. Cl. C12Q 1/68; C07H 21/04

(52) U.S. Cl. 435/6; 435/270; 536/25.4

(57) ABSTRACT

A method for isolating RNA, DNA or proteins from an animal tissue sample that is amenable to high throughput adaptation is disclosed. The method involves beadmilling the sample to disrupt the cells contained therein and extracting RNA, DNA or proteins from the disrupted sample by solid phase extraction.

### STATEMENT REGARDING FEDERALLY SPONSORED RESEARCH OR DEVELOPMENT

This invention was made with United States government support awarded by the following agency: Department of Energy, Contract No. DE-AC05-00OR22725. The United States has certain rights in this invention.

### CROSS-REFERENCE TO RELATED APPLICATIONS

Not Applicable.

### BACKGROUND OF THE INVENTION

The study of gene expression has changed to a high-throughput science as gene-chip microarray technology has replaced conventional modes of expression analyses. Microarrays contain miniaturized spots of gene-definitive cDNAs or oligomers (probes) arranged into a grid and interrogated with a target RNA (actually a labeled ssDNA copy of the. RNA pool, produced by reverse transcriptase). The target RNA represents the expressed genes of a given tissue-type at some time point, and the genes expressed are identified by hybridization to the probes. Labeling methods vary, but commonly involve fluorophores incorporated into the cDNA during the reverse transcriptase reaction. To compare expression patterns between cell types, or time points, each target is labeled with a different fluorophore. By mixing the differently labeled targets during a competitive hybridization (target limiting) the relative abundance of transcripts, and therefore the relative expression level of individual genes, can be determined. This technology enables the simultaneous analysis of thousands of expressed genes and is already elucidating the complex metabolism of cells in unprecedented detail.

Traditional RNA extraction using high-speed mechanical disruption in the presence of chaotropic salts, followed by acid-phenol purification or CsCl buoyant density centrifugation is generally considered to produce the highest quality RNA from tissues. But this is a labor-intensive procedure that involves extraction with organic solvents, and rate-limiting ultracentrifuge runs. These processes involve considerable hands on manipulation increasing the chance for RNase contamination. Further, the enzyme RNase is present intracellularly in tissue samples, and so samples must be handled carefully, and quickly, before and during the disruption process. Typically, fresh tissues must be processed within minutes or seconds of harvest. If a number of tissues must be harvested, the tissues are usually snap-frozen in liquid nitrogen to prevent RNA degradation. Unfortunately, freezing also exacerbates the processing of tissues, as they must be ground while still frozen, and thawed in the presence of strong RNase-inhibiting compounds. This effectively limits the breadth of experimentation. When using valuable animal models, all available tissues cannot be harvested, largely because of the difficulty in processing large numbers of samples, or because of RNA degradation in harvested tissues that are awaiting processing.

As the cost of genechips decreases, thousands of microarray experiments can be performed in a cost-efficient manner. Traditional centrifugation isolation methodologies for RNA purification are unlikely to produce the throughput needed for these experiments. While high throughput solid-phase methods for isolating high-quality RNA exist, they usually start with lysed tissues obtained through manual preparations, which perpetuate a bottleneck in the production of RNA from animal tissues and result in a very low-throughput. Beadmilling represents a high throughput closed system capable of incorporating tissue processing into an automation scheme. However, only organic-extraction methods, which are difficult to set-up as high throughput, have been shown to be able to extract RNA from beadmilled tissues. What is needed in the art is a method of isolating RNA from harvested animal tissues that is amenable to high throughput adaptation.

### SUMMARY OF THE INVENTION

In one aspect, the present invention provides a method for isolating RNA, DNA or protein from an animal tissue sample. The method involves beadmilling the sample in a beadmilling buffer to disrupt cells in the sample and extracting RNA, DNA or protein from the sample using a solid phase extraction (SPE) method.

In one embodiment, the method of the present invention is used for isolating RNA from an animal tissue sample. The method involves preserving the tissue sample in a RNA preservation buffer, switching the tissue sample to a beadmilling buffer that contains a ribonuclease inhibitor, beadmilling the tissue sample in the beadmilling buffer to disrupt cells in the sample, and extracting RNA from the tissue sample using a solid phase extraction method. The beadmilling conditions in this embodiment are such that the DNA released from the disrupted cells is sufficiently sheared for solid phase RNA extraction.

It is an advantage of the present invention that the method is amenable to high throughput adaptation.

It is another advantage of the present invention that many commercially available agents and kits can be used in connection with the method of the present invention.

Other advantages, features and objects of the invention will become apparent from the following detailed description when considered in conjunction with the accompanying claims and drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### BRIEF DESCRIPTION OF THE DRAWINGS

FIG. 1 shows comparison of CsCl-prepared total RNA to QIAGEN (Valencia, Calif.) SPE-prepared RNA. Lanes A-C show 5 μg mouse liver total RNA prepared by CsCl buoyant density centrifugation after disruption of the tissue using a polytron homogenizer. Lanes D-F show 5 μg of mouse liver RNAs produced using QIAGEN's RNAeasy columns, using polytron disrupted tissue. RNA samples were electrophoresed in a non-denaturing 1.5% agarose gel.

FIG. 2 shows the effect of bead size, bead type and cycling number on the beadmilling process. Twenty-five mg of mouse skin was disrupted using the FastPrep™ (FP120, BIO 101) shaker. The left two tubes in each panel contained 3 mm glass beads. The middle two tubes of each panel contained 1 mm glass beads, and the right two tubes in each panel contained 1.5 mm ceramic beads. Each tube contained 25 mg skin tissue. After cycling, tubes were briefly centrifuged (30 seconds at 13,500 rpm), to clear the liquid for visualization of undispersed tissue. Panel A shows the tubes before cycling. Panels B, C and D show the tubes after 3, 6 and 9 cycles, respectively.

FIG. 3 shows the effect of bead number on the beadmilling process. The panels A, B, C, and D show the results of experiments using 0, 2, 4, and 6 cycles, respectively. The numbers of beads used were 0, 2, 4, 6, 8, and 10 in the tubes shown (left to right).

FIG. 4 shows the effect of increased cycling on DNA shearing in vitro. Disruption tubes with six 3 mm glass beads and containing either 500 μl (top row) or 1,000 μl (bottom row) of 2 μg/μl calf thymus DNA were cycled 14 times in a FastPrep shaker. After each cycle, 2 μl aliquots were removed and electrophoresed in a non-denaturing agarose gel, and stained with EtBr for visualization. The left hand lane of each panel shows the control unsheared DNA before cycling. Lane numbers indicate the number of cycles for each sample. Lambda DNA digested with HindIII restriction enzyme was used as a molecular weight marker.

FIG. 5 shows the effect of increased cycling on shearing DNA in tissue. Mouse skin samples (25 mg) were disrupted in 1,000 μl disruption buffer and RNA was isolated oil a QIAGEN RNAeasy column, then visualized by electrophoresis and EtBr staining. Lane A: mouse skin RNA isolated after tissue was cycled five times. Lane B: mouse skin RNA isolated after tissue was cycled 15 times.

FIG. 6 shows the effect of disruption volumes on RNA quality using mouse skin and mouse liver tissues. Either 1,000 μl (Lanes A and C) or 500 μl (Lanes B and D) of disruption volume were used in disruption tubes with six 3 mm glass beads. Mouse skin (Lanes A and B) and liver (Lanes C and D) tissues were disrupted and RNA was isolated and visualized as described in previous figures. Liver preparations were overloaded but showed intact 28S RNA bands of identical density after longer electrophoresis (data not shown).

FIG. 7 shows the effect of lysis buffer on RNA quality using beadmilling. Lanes A and B: RNA preparations from mouse skin using guanidium isothiocyanate (GITC) as disruption buffer; Lanes C and D: RNA preparations from mouse skin using the RLE disruption buffer supplied with the QIAGEN RNAeasy kits.

FIG. 8 shows 12 optimized RNA preparations from mouse skin tissues using beadmilling and minicolumns. The tissues were beadmilled for a total of 15 cycles (cooled on ice after every 5 cycles) in disruption tubes containing six 3-mm glass beads and 1,000 μl of GITC disruption buffer. RNA was isolated on minicolumns, and visualized by native agarose gel electrophoresis and EtBr staining.

FIG. 9 shows the electropherogram of 96 mouse liver RNAs produced using optimized beadmilling and adapted for an automated RNA extraction process using 96-well format. Each lane contains 1/20 of the liver RNA from a 30 mg sample.

FIG. 10 shows the microarray results using RNAs produced by beadmilling and solid phase extraction. Panel A: Mouse skin (Red-Cy5) and mouse liver (Green-Cy3, pooled from the samples shown in FIG. 9) hybridized to mouse cDNAs. Known liver-specific genes are indicated with small white boxes. Panel B: Zebrafish embryo RNA from toxin-treated (Red-Cy3) or untreated (Green-Cy5) samples hybridized to zebrafish cDNAs. Known toxic-response genes are indicated with white boxes. Panel A used 3×SSC as spotting buffer, while panel B used 50% DMSO as spotting buffer.

### DETAILED DESCRIPTION OF THE INVENTION

The term “animal tissue sample” used in the specification and claims encompasses tissue samples from both non-human animals and humans. In the specification and claims, the term “about” has been used before certain numbers that describe glass bead size, beadmilling solution volume to glass bead volume ratio, temperature, shaking cycle time and shaking speed. Used as such, the term “about” along with the stated number encompass small deviations from the stated number that do not alter the essential functionality of that number.

The present invention relates a method for isolating RNA, DNA and proteins from an animal tissue sample that is amenable to high throughput adaptation. The method involves two high throughput adaptable steps: beadmilling the tissue sample (shaking the tissue sample in the presence of beads) to disrupt tissues and cells and extracting RNA, DNA or protein by solid phase extraction.

The present invention is based on the inventors' endeavor of developing an automated procedure for RNA isolation from animal tissues to suit the need of high throughput microarray-based assays. Critical factors that can affect the quality and success of RNA isolation from a biological sample include RNA degradation by ribonucleases contained in the biological sample and interference of RNA extraction by certain components (e.g., genomic DNA) of the biological sample. RNA isolation from a biological sample usually involves a two step procedure of disrupting the sample and extracting RNA from the disrupted sample. For successful RNA isolation, the sample disruption step should not only disrupt the sample to cause the release of RNA but also retain sufficient amount of RNA from ribonuclease degradation and disrupt other components of the sample that interfere with the next step of RNA extraction. Since different types of biological samples vary in the amount of ribonucleases and the type and amount of other components that interfere with RNA extraction, it is crucial that a specific sample disruption method that can achieve the above objectives of the step is selected for disrupting a particular type of biological sample. For animal tissues, the method selected has been either homogenization by a polytron homogenizer or freezing and then grinding with a mortar and a pestle. Since both polytron homogenization and freezing and mortar-pestle grinding are labor-intensive, it has been difficult to isolate RNA from animal tissues in a high throughput manner. While beadmilling is a known tissue disruption technique that can be readily automated, it has only been recommended and used for yeast cells for RNA isolation. What the inventors have demonstrated here is that when used adequately, beadmilling, which has not been recommended for RNA isolation from animal tissues, can produce disrupted animal tissues suitable for RNA extraction by a solid phase method. In combination with the demonstration that RNA in animal tissues can be preserved in an RNA preservation solution such as RNAlater™ (Ambion Inc., Austin, Tex.) at a non-freezing temperature (e.g., room temperature), the inventors have established a method of RNA isolation from animal tissues that can be adapted for high throughput procedures.

Although the method is developed by the inventors to isolate RNA from animal tissues, it can also be used for isolating DNA or proteins from animal tissues. Specifics of the method for DNA or protein isolation can be readily determined by a skilled artisan. Custom protocols and commercial agents and kits suitable for use in the method of the present invention for RNA isolation are described in the specification. Similar custom protocols and commercial DNA and protein agents and kits are available and can be used for DNA and protein isolation.

Using RNA isolation as an example, the method of the present invention is described in greater detail below. Depending on the tissue type and amount, it is understood that preferred parameters (e.g., those for bead size, buffer volume, shaking speed and shaking time) as described below can be adjusted for optimal results.

The starting material for the method of the present invention is an animal tissue sample. The amount of tissue that can be beadmilled for RNA extraction is largely determined by the capacity of the downstream solid phase method. When the tissue sample contains relatively large tissue masses (e.g., 0.5 cm³ or larger), it is preferable to mince the tissues to smaller sizes (e.g., less than 0.5 cm³). For RNA isolation, necessary steps need to be taken to reduce RNA degradation caused by internal (intrinsic) and external (extrinsic) ribonucleases throughout the tissue harvest and RNA extraction process. A skilled artisan is familiar with these steps. For example, ribonuclease inhibitors can be used in the method of the present invention. Examples of ribonuclease inhibitors can be found in Farrell R. E. (ed.) (RNA Methodologies: A Laboratory Guide for Isolation and Characterization, Academic Press, 1993) and Jones, P. et al. (In: RNA Isolation and Analysis, Bios Scientific Publishers, Oxford, 1994), each of which is herein incorporated by reference in its entirety. The most commonly used ribonuclease inhibitors include guanidium hydrochloride and guanidium thiocyanate. As strong ionic protein denaturants, often referred to as chaotropic reagents, these two compounds can also help lyse cells. If a sample will not be immediately used for RNA extraction, freezing the sample can also help preserve RNA. Steps that can be taken to minimize external ribonuclease contamination include the use of ribonuclease-free glassware and plasticware and the use of ribonuclease inhibitors such as diethylpyrocarbonate (DEPC) and vanadyl compounds.

Recently, a proprietary ribonuclease-inhibiting agent, RNAlater™ (Ambion Inc., Austin, Tex., U.S. Pat. No. 6,204,375, which is herein incorporated by reference in its entirety), has been shown to be able to stabilizing RNA in situ (within tissues) and to protect the RNA during the extraction process. As shown in the example below, biological samples collected for RNA isolation can be preserved in RNAlater™ at room temperature for a long time without losing significant amount of RNA. This is especially helpful in high throughput applications in which a large number of samples need to be harvested and the time between the harvest of the first sample and the last sample can be relatively long so that significant amount of RNA may be degraded. A conventional method to prevent this RNA degradation is to fast freeze the samples. The freezing step can now be eliminated by preserving the samples in RNAlater™ until ready for the beadmilling step. It is understood that other RNA preservation agents that are similar to RNAlater™ can also be used in the method of the present invention.

The type of beads that can be used in the method of the present invention for RNA isolation include but are not limited to glass beads and ceramic beads. Glass beads are preferred and the size of the glass beads (in diameter) can be from about 2 mm to about 5 mm, from about 3 mm to about 4 mm, or preferably about 3 mm.

The beadmilling step should be conducted in a buffer (beadmilling or shaking buffer, also referred to as disruption or lysis buffer in the specification) that contains a ribonuclease inhibitor. A skilled artisan is familiar with the buffers that can be used for the beadmilling step. For example, the guanidium isothiocyanate disruption buffer described in Chomczynski, P. and Sacchi, N. (Analytical Biochemistry 162:156-159, 1987, which is herein incorporated by reference in its entirety) is a suitable buffer. The amount of buffer used is determined by the total volume of beads used and vise versa. Preferably, the buffer to beads ratio (volume/volume) is from about 1.3/1 (calculated by 500 μl buffer to six 5-mm beads) to about 79.6/1 (calculated by 2,000 μl buffer to six 2-mm beads), from about 2.5/1 (calculated by 500 μl buffer to six 4-mm beads) to about 23.6/1 (calculated by 2,000 μl buffer to six 3-mm beads), from about 2.6/1 (calculated by 1,000 μl buffer to six 5-mm beads) to about 39.8/1 (calculated by 1,000 μl buffer to six 2-mm beads), from about 5.0/1 (calculated by 1,000 μl buffer to six 4-mm beads) to about 11.8/1 (calculated by 1,000 μl buffer to six 3-mm beads), or about 11.8/1 (calculated by 1,000 μl buffer to six 3-mm beads).

In order for a tissue sample to be sufficiently disrupted, several beadmilling cycles (shaking cycles) may be necessary. For RNA isolation, the preferred number of shaking cycles is from 5 to 20 cycles, from 6 to 9 cycles, or from 14 to 15 cycles. The preferred shaking time for each cycle is from about 30 seconds to about 60 seconds. The most preferred shaking time for each cycle is about 45 seconds. Also preferably, the sample is cooled every 1 to 5 shaking cycles. Most preferably, the sample is cooled every 5 shaking cycles. The temperature to which the sample is cooled is between 0° C. and 25° C. Preferably, the sample is cooled to about 4° C.

A variety of motions can be employed either alone or in combination to beadmill a sample. For example, a high speed shaker using minute throw and yaw vectors can be used to beadmill a sample. No matter what motions are employed, shaking speed for beadmilling the sample is preferably from about 3 meters per second to about 10 meters per second, or from about 5 meters per second to about 8 meters per second. The most preferred shaking speed is about 6.5 meters per second.

After tissue disruption, a solid phase extraction method is employed in the present invention to extract RNA. Solid phase extraction of biomolecules including RNA, DNA and proteins are mature technologies that a skilled artisan is familiar with. A variety of commercial products are available for this purpose and can be used in the present invention.

An advantage of the method of the present invention is that it can be easily adapted to high throughput processes. As an example, a high throughput tissue-to-RNA process may involve the following steps: adding RNAlater™ into 96-well plates, harvesting tissues into the 96-well plates, replacing RNAlater™ with a beadmilling buffer in the 96-well plates, adding glass beads into the 96-well plates, sealing the 96-well plates, shaking the 96-well plates (beadmilling), and extracting RNA by solid phase extraction. 96-well plates and plate lids constructed of either polypropylene or silicon that is both leak proof and strong enough to withstand beadmilling are commercially available. Solid phase extraction of RNA can be automated easily and a variety of commercial products for solid phase extraction are available in high throughput format (e.g., QIAGEN's RNAEasy96-kit). A robotic system capable of shaking and liquid handling can be readily assembled using a shaker and a versatile liquid handling robot such as one similar to MultiProbeII from Perkin Elmer (Boston, Mass.). Thus, with the exception of tissue harvesting, the whole tissue-to-RNA process can be readily automated with a robotic system capable of liquid handling and shaking. As a result, the bottleneck in processing RNA for targets is effectively moved to the tissue harvest step. The 96-well process allows several hundred samples be processed simultaneously.

Tissues that are less dense than RNAlater™ (e.g. fish embryos) will float in the solution and thus create a problem for automating the RNAlater™ removing step in a high throughput application (floating does not adversely affect RNA preservation in RNAlater™. For these tissues, RNAlater™ can be safely diluted with an equal volume of ice-cold ribonuclease-free water, which allows the tissues to be rapidly pelleted and the RNAlater™ aspirated or decanted. The tissues can then be resuspended in a beadmilling buffer for beadmilling. It is possible that this dilution may lead to reactivation of ribonucleases by allowing it to resuspend. Thus, time spent in the diluted preservative is preferably kept as cold and short as possible.

The invention will be more fully understood upon consideration of the following non-limiting example.

### EXAMPLE

Materials and Methods

Reagents and Supplies: Clones were selected from either the NIA 15K clone set (Tanaka et al.), #NIAH3000VU-11, average cDNA insert size of 1.6 KB, pSPORT1 vector, E. coli host strain DH10B, purchased from Incyte Genomics, Inc. (Palo Alto, Calif.) or from Research Genetics (Huntsville, Ala.). QIAprep 96 Turbo Miniprep Kits (cat. #27191), QIAGEN QIAquick 96 PCR purification kit (QIAGEN cat. #28181) and related reagents were purchased from QIAGEN, Inc. (Valencia, Calif.). PCR reagent kits were purchased from Perkin Elmer/Roche (Branchburg, N.J.) and Promega Corp. (Madison, Wis.). Primers NIA1 (5′-GTTTTCCCAGTCACGACGTTG-3′) (SEQ ID NO:1) and NIA2 (5′-TGAGCGGATAACAATTTCACACAG-3′) (SEQ ID NO:2) were purchased from Integrated DNA Technologies, Inc. (Coralville, Iowa). Superscript II Reverse Transcriptase was from Bethesda Research Laboratories (Bethesda, Md.).

Conductive, liquid sensing pipette tips were purchased from Packard BioSciences (Meriden, Conn.) or Molecular BioProducts (San Diego, Calif.). Chemicals and reagents for molecular biology or probe production were purchased from Sigma Chemical Co. (St, Louis, Mo.). Plasticware was purchased from various manufacturers including: standard 96-well microtiter assay plates (#3896, Corning Costar Co., Cambridge, Mass.), deep well 96-well plates for growing bacteria (Whatman 2-ml Uniplate #7701-5205), 384-well polypropylene plates for spotting (Whatman Polyfiltronics Uniplate #7701-5101), black 96-well fluorescence reader plates (Dynatech microfluor® 1, Thermo Labsystems, Helsinki, Finland). Standard molecular biology protocols were followed when appropriate (Sambrook, et al.).

Instrument Description/Configuration: The most current description of the Packard Instruments MPII HT EX with Gripper Integration Platform and Nucleic Acid Extraction (NAE) Application Option can be found with the manufacture. This system is developed to fully automate plasmid DNA purification using a variety of commercially available kits and filter plates. In this example, empty 1.2 ml collection tube racks were modified and implemented as spacers for elution into standard microplates.

Plasmid Preparation: Bacterial stocks were replicated using a 96-pin tool capable of 25 ™ l liquid transfers (V&P Scientific, San Diego, Calif., #VP408), into 96-well deep well growth plates containing 1.5 ml of 2×YT bacterial media with glycerol (Tryptone; 16.0 g/l, yeast extract; 10.0 g/l, NaCl; 5.0 g/l, 0.1 M sodium citrate; 17.0 g/l, 1.0 M K₂HPO₄; 36.0 ml/l, 1.0 M KH₂PO₄; 13.2 ml/l, 80% glycerol; 55.0 ml/l, 1.0 M MgSO₄; 0.4 ml/l, 2.0 M (NH₄)₂; 36.0 ml/l, 3.4 ml/], pH=7.0) and the appropriate antibiotic (100 μ/ml ampicillin). Bacteria were grown 16 hours at 37° C. with shaking (250 RPM). Subsequently, the bacterial cells were pelleted by centrifugation in a Sorvall RC5B with Sorvall SH3000 rotor at 5000×g for 15 minutes, after which the plates are inverted and blotted to remove excess media. Automated nucleic acid plasmid DNA purification was preformed using the QIAGEN QIAprep® 96 Turbo Miniprep Kit. A sample elution volume of 100 μl of EB buffer was used.

PCR Product Preparation: PCR reactions (50 μl) used for microarray probe production contain 5 μl 10× reaction buffer, 4 μl 25 mM MgCl₂, 1 μl 10 mM dNTPs, 1 unit Taq DNA polymerase, 100 ng each of the appropriate primers and 1 μl of plasmid DNA (approximately 200 ng). Depending on the particular cloning vector used for cloning, a different master mix was prepared containing the correct pair of oligonucleotide amplimers. Amplification was performed using in a GeneAmp 9600 thermocycler (Perkin Elmer/Roche, Branchburg, N.J.). PCR conditions are: 95° C. for 5 minutes followed by 40 cycles of 94° C. 30 seconds, 54° C. 30 seconds, and 73° C. 3.5 minutes, finished with seven minutes at 72° C.

PCR products were purified from excess primers and nucleotides using the MultiPROBE II workstation and the QIAGEN QIAquick™ 96 PCR purification kit with supplied buffers and labware. After purification, the PCR products were covered loosely and dried overnight at 50° C. The dried PCR products were resuspended in the appropriate volume (determined by DNA quantitation, see below) of 3×SSC (Standard Saline Citrate buffer: 150 mM NaCl, 15 mM NaCitrate, pH 7.0) buffer to prepare approximately 150 μg/ml solution. These “chip-ready” probes were stored frozen at −80° C., in 96-well format, until needed to prepare the microarrays. For microarray construction the DNA's were re-distributed into 384-well format using the MultiPROBE II.

Microarray Production and Analysis: Microarrays were analyzed essentially as described in Hedge, et al. To “spot” DNA arrays, PCR products were thawed and gently vortexed prior to placement onto the deck of a VIRTEK (Ontario, Canada) Chip Writer (model SDDC-2) fitted with 4 Arrayit™ Stealth MicroSpotting pins (Telechem International Inc., Sunnyvale, Calif.). Arrays were produced onto CMT-GAPS™ amino-silane coated microscope slides (cat #2550, Coming Inc., Corning, N.Y.). The arrays were dried, post-processed according to Hegde et al., and stored in a desiccator until use. Immediately prior to application of the target, the slides were prehybridized for 1 hour at 42° C. in BSA. The slides were then briefly rinsed in water and dried in a stream of compressed air or nitrogen.

RNA Labeling: Hybridization targets (labeled cRNA) were produced using Superscript II reverse transcriptase (#18064-022, Life Technologies, Rockville, Md.), 25 to 80 μg of total RNA and Cyanine 3-dUTP (#NEL578, Perkin Elmer, Boston, Mass.) and Cyanine 5-dUTP (#NEL579, Perkin Elmer, Boston, Mass.) fluorophores (Tanaka et al., 2000). Labeled cRNA was subsequently mixed with 10 μg of poly[dA] plus 10 μg of murine Cot-1 DNA (#18440-016, Life Technologies, Rockville, Md.), dried, then resuspended in 20 μl of hybridization buffer containing 50% formamide, 4×SSC, and 0.1% SDS. The cRNA mixture was applied to the array, covered with a No. 1 (22×40 mm) cover glass (Corning, Cambridge, Mass.), and placed in a fully humidified and sealed chamber at 42° C. for a minimum of 16 hours. After hybridization, the arrays were washed once with 2×SSC, 0.1% SDS at 42° C. for 5 minutes, twice in 0.2×SSC at room temperature for 2 minutes, and once in 0.1×SSC at room temperature for 1 minute. The arrays were then blown dry using a stream of compressed air or nitrogen. Slides were imaged using a Packard BioSciences ScanArray 4000XL microarray confocal laser scanner at 5 or 10 micron resolution.

Tissue Preparation: Tissues were harvested by rapid dissection and placed into ≧5 volumes of RNAlater™ at 0° C. Larger tissues were first rapidly minced to less than 0.5 cm³. Tissues were then stored 16-72 hours at 2-4° C. in 5 volumes of RNAlater™ to completely stabilize the RNA. Longer storage was at 20° C., where samples were tested up to 1 year later and the RNA found to be intact. After these treatments, all other manipulations of the tissue were performed at room temperature unless specifically stated otherwise.

RNA Production: Tissues were placed into milling tubes which are 1.5 ml screw-cap microcentrifuge tubes with O-ring caps (Bio Plas, Inc., #4202 and #4215R) along with seven, borosilicate 3 mm solid glass beads (Aldrich #Z14392-8, Milwaukee, Wis.), and 500-1,000 μl of guanidium isothiocyanate disruption buffer (Chomczynski and Sacchi 1987). The tissues were disrupted, and the DNA homogenized (sheared) by shaking at high speed (6.5 meters/second) in an FP120 FastPrep™ Cell Disruptor (BIO 101, Vista, Calif.). Times for complete disruption varied depending on the type of tissue. Usually, 10 to 15 cycles of 45 seconds with cooling to 0° C. after every 5 cycles, were used with good results. Beads were allowed to settle for 1 hour at 4° C., or the tubes can be centrifuged to reduce foaming. Fully disrupted tissues were loaded automatically onto commercial solid-phase RNA extraction columns (RNAeasy™ l, QIAGEN Inc., Valencia, Calif.), using a Packard Multiprobe™ II Robotic liquid handling system with Nucleic Acid Option. Samples were processed according to the manufacturers instructions, Using a custom control program written using the MultiProbe WinPrep™ software. The QIAGEN QIAvac 96 vacuum manifold (#19504) was used with the Packard manifold adapter plate.

DNA Shearing: Genomic DNA (Calf Thymus) at 2 μg/l was placed into a milling tube, containing six 3 mm glass beads, and milled at 6.5m/second for 45-second cycles. A total of 14 cycles were examined. Two μl of sample was removed at the end of every cycle for evaluation. In separate experiments either 1,000 μl or 500 μl of starting material was used to compare the effect of volume on beadmilling. The removed samples were electrophoresed through an agarose gel, stained, and visualized to determine the presence of intact genomic DNA, and to estimate the overall size of the DNA.

Results

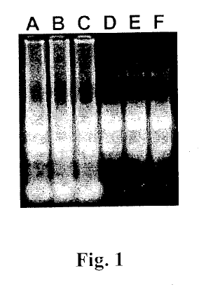

RNAlater™ preservation of RNA in tissues: We have tested the quality and quantity of RNA from the following mouse tissues preserved in RNAlater™: skin, liver, lung, brain, muscle, kidney, lymph node, fat, heart, spleen, gut, testes, thyroid, lung, solid tumor, tail clip, and ovary. In all cases, the RNA observed was undegraded. Also, excellent RNA preservation results were obtained with whole adult zebrafish, adult zebrafish ovaries, and embryonic zebrafish at days 1, 2, 5, and 7 or gestation. Our results also showed that mouse tissues extracted into RNAlater™ and isolated using QIAGEN RNAeasy spin columns were not degraded and easily comparable to the quality reached using fresh tissues lysed using a polytron homogenizer and isolated using all acid-phenol extraction protocol (Chomczynski and Sacchi 1987) (FIG. 1). Extensive characterization of RNA isolated by these resins can be-found with the manufacture QIAGEN Inc.

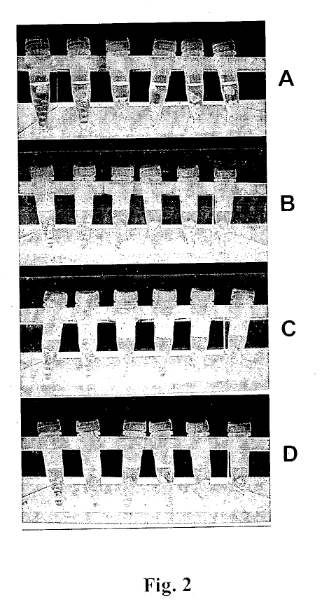

Comparison of bead type: To determine the best type of bead to use in the milling process, several variations of beads were tested. FIG. 2 shows the results of using three different bead types on the milling process. For this representative experiment, we chose 230 mg of beads, because this approximated the optimal amount (½ to ¼ the volume of the disruption buffer, see below). The 3 mm glass beads showed the greatest ability to disrupt the tissues when compared to smaller glass or ceramic beads, in this shaking configuration. Further, between 6 and 9 cycles was the minimum to disrupt the tissues. Disruption was tissue-type dependent, and soft tissues generally require less disruption to disperse their cells, while fibrous or tough tissues like skin required more.

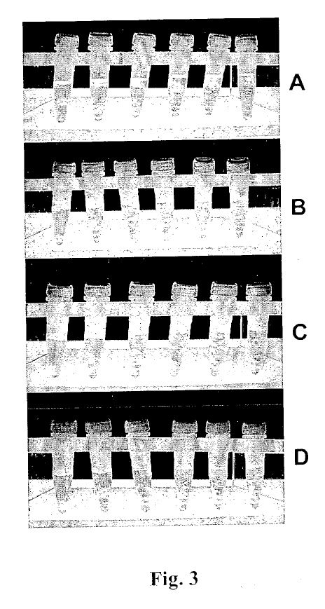

To determine the optimal number of beads that could be used in these experiments, we performed an experiment similar to that in FIG. 2 using 3 mm glass beads, and varying the number of beads within each tube. The results of these experiments are shown in FIG. 3. These results showed that the tissues were completely disrupted after 6 cycles using 6 or more glass beads per tube. The six beads correspond to approximately 230 mg. These combined experiments indicate that the minimum conditions for tissue disruption are six, 3 mm glass beads in 0.5 ml lysis buffer (also referred to as beadmilling buffer), for six 45 second cycles at 6.5 m/second.

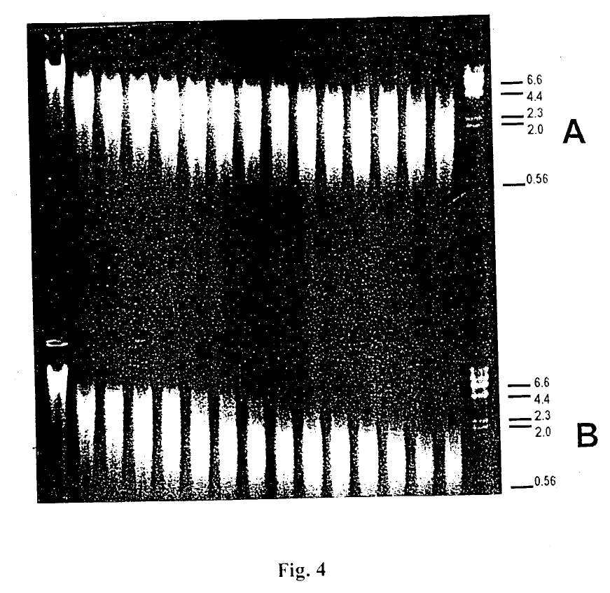

Genomic DNA shearing: QIAGEN RNAeasy columns require that the high molecular weight DNA be sheared to prevent DNA appearing in the column elution. To determine the optimal conditions for DNA shearing, genomic DNA was placed into milling tubes, and observed for shearing after each cycle (FIG. 4). Using 1,000 μl of DNA in solution, only 7-8 cycles were required to eliminate high molecular weight DNA from the tube, and reduced the average DNA fragment length to less than 2 kb. Conversely, with the 500 μl DNA samples, a band of high molecular weight DNA persisted through 8 cycles, and did not completely disappear until 14 cycles. The average molecular weight appeared to be similarly resistant to cycling, only dropping below the 2 kb size at 14 cycles. These results indicate that a 1,000 μl starting volume was more efficient at shearing DNA than using 500 μl under these conditions.

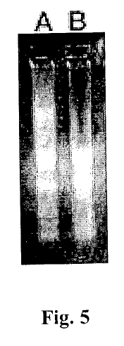

Similar effects of increased cycling on DNA shearing were observed using mouse skin tissue samples. As shown in FIG. 5, 5 cycles of shaking resulted in residual genomic DNA contamination of the RNA. Conversely, after 15 cycles (with cooling after every 5 cycles), the genomic DNA was eliminated from the eluant. Cooling on ice after every 5 shaking cycles was found to significantly improve the RNA quality and yield.

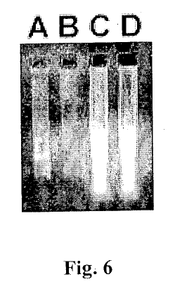

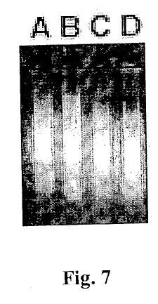

Effect of lysis volume on RNA quality: In FIG. 6, a comparison of 500 μl and 1,000 μl volumes was performed using skin and liver tissues. When 500 μl of disruption buffer was used, RNA was occasionally isolated from mouse skin that lacked a 28S RNA band, suggesting that the RNA was degraded. Although the absolute yields from the two tissues were dramatically different, FIG. 6 shows that skin tissue required a larger disruption buffer volume, while liver was not as sensitive to disruption volume. Without intending to be limited by theory, we interpret these data to indicate that either the RNAlater™ preservative, or intrinsic cellular components, influences the functionality of the lysis buffer, and increasing the overall volume minimizes these influences. In agreement with this theory, we found the choice of lysis buffer could also affect RNA quality, as indicated by FIG. 7. Homemade guanidium lysis buffer (Chomczynski and Sacchi 1987) was better for RNA production with the preserved tissues than the lysis buffer (“RLT”) supplied with the RNA extraction kit (QIAGEN).

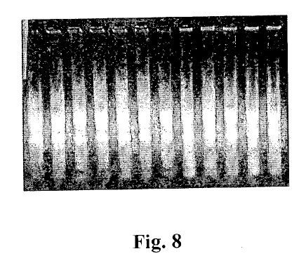

Final optimization: The result of these optimization processes can be seen in FIG. 8 for twelve mouse skin RNA preparations where the overall quality and consistency of the RNA preparations was evident. The average RNA yield from these RNAs, was 20.1 (±5.6 S.D.) μg/30 mg tissue, or 650 μg/g tissue (FIG. 8). These values are well within the expected yields for these columns. The average A260/A280 ratio for these RNAs was 2.0 (±0.19 S.D.).

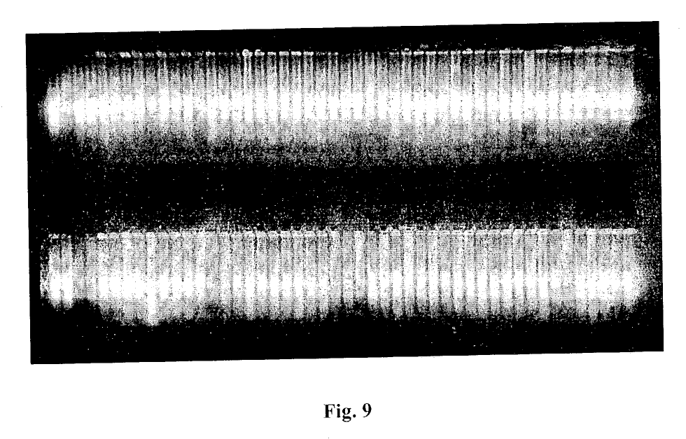

Robotic Processing: An ultimate goal for high-throughput RNA production is the ability to use 96-well formats which ameliorate robotic processes and sample tracking automation. We developed protocols to produce 96 RNA samples in a few hours using our optimized protocol and a Packard Multiprobe II Liquid handling robot. WINPREP software controls were written to perform automated RNA purification using the Multiprobe according to the RNAeasy-96 manufacturer's instructions. Using mouse liver as an example, 96 samples were processed within 3 hours with a semi-automated procedure. The success rate was 99% (95/96 samples undegraded, FIG. 9). These experiments used approximately 30 mg of tissue per milling tube (visually estimated) and a vacuum manifold to dry columns between process steps. The variations in visual estimates, and the possibility of wash solutions persisting in the column tips during vacuuming steps, likely account for the variations in the yield and quality of the RNAs seen. The overall yield for these samples was 3.1 mg/g tissue.

Use in microarrays: We have used RNAs as targets in microarray experiments to determine differential gene expression. Our initial RNA quality controls relied on high throughput adaptations of conventional gel-electrophoresis techniques to visually evaluate the samples. We adapted the Packard Multiprobe to load up to 192 cDNA or RNA samples from 96-well microtitre plates onto a submerged gel in 30-40 minutes. Additionally, a 96-well spectrophotometric plate reader (Perkin Elmer) allowed us to make quantitative measurements of the nucleic acid concentrations and purity.

All our microarrays include heterologous control probes used to normalize for hybridization efficiency and probe or target functionality. On microarrays, our beadmill-produced RNAs have been compared tissue-to-tissue with known high-quality RNA targets produced by conventional processes and no significant difference was observed.

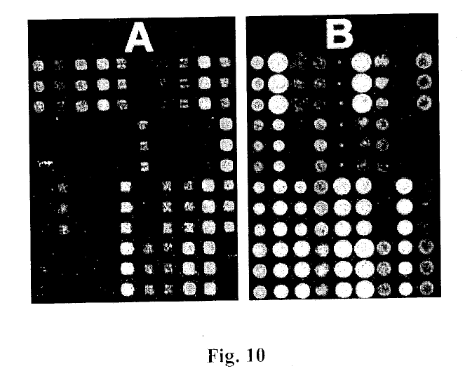

An example of the results obtained using RNAs prepared by our semi-automated processes and used to interrogate cDNA microarrays is shown in FIG. 10. Using mouse skin and liver RNA (Panel A) hybridized to cDNA microarrays, the high quality and reproducibility of the hybridization allowed for the quantification of differential expression of many genes.

Extraction of RNA using solid-phase supports is a mature technology. Several commercially available techniques and kits are available for these procedures, many available in a high-throughput (96-well or greater) system. Any 96-well format kit that can use a vacuum manifold for RNA isolation should perform acceptably using the setup described in this example, unless the kit buffers are unusually sensitive to the components of RNAlater™. The QIAGEN RNAeasy kits were used primarily because they were robust, and showed resistance to clogging when used with different mouse tissues. The appearance of RNA produced using a vacuum manifold, as opposed to using centrifugal systems, was slightly poorer, based on the overall appearance of streaking within the RNA lane (compare FIG. 9 to FIG. 8). However, these RNAs were not degraded, and performed well in microarray experiments. Some of these differences could be due to the length of time required to load gels when examining 96 samples. We have examined methods to reduce streaking in vacuum-manifold prepared RNAs and found that better drying of the columns during wash and prior to elution was helpful. Although this required user intervention, we resorted to pausing the purification process after the wash steps, to manually blot the QIAGEN extraction plate. This resulted in a cleaner, more consistent eluant. Packard has implemented an automated blotting process in their current Multiprobe instrumentation processes, which should be sufficient to allow walk-away automation to proceed.

The present invention is not intended to be limited to the foregoing example, but encompasses all such modifications and variations as come within the scope of the appended claims.

References

Chomczynski, P. and Sacchi, N., 1987. Single-step method of RNA isolation by acid guanidinium thiocyanate-phenol-chlorofirm extraction. Analytical Biochemistry 162:156-159

Chirgwin, J. M., et al. (1979) Biochemistry 18: 5294-5299

Hegde, P., Qi, R., Abernathy, K., Gay, C., Dharap, S., Gaspard, R., Earle-Hughes, J., Snesrud, E., Lee, N., and Quackenbush, J., A Concise Guide to cDNA Microarray Analysis. Biotechniques, 29(3), September 2000,548-562

Sambrook, J., Fritsch, E. F., Maniatis, T. Molecular Cloning. A laboratory manual. Second Edition. Cold Spring Harbor, N.Y.: Cold Spring Harbor laboratory Press, 1989.

Packard Specification Bulletin: Talon ™ Integration Platform. Nucleic Acid Extraction Application. No M4197 January, 2001. Packard BioScience Company. Packard Liquid Handling. Application Note LHN-002. Plasmid Purifications. Packard BioScience Company.

Tanaka, T. S., A. Jaradat, M. K. Lim, G. J. Kargul, X. Wang, M. J. Grahovac, S. Pantano, Y. Sano, Y. Piao, R. Nagaraja, H. Doi, W. H. Wood III, K. G. Becker, M. S. H. Ko, Genome-wide expression profiling of mid-gestation placenta and embryo using a 15,000 mouse developmental cDNA microarray. Proc. Natl. Acad. Sci., 97(16), August, 2000.

Engelstein M, Aldredge T J, Madan D, Smith J H, Mao J I, Smith D R, Rice P W. An efficient, automatable template preparation for high throughput sequencing. Microb Comp Genomics 1998;3(4):237-41

Neudecker F, Grimm S. High-throughput method for isolating plasmid DNA with reduced lipopolysaccharide content. Biotechniques January 2000;28(1):107-9

Skowronski E W, Armstrong N, Andersen G, Macht M, McCready P M. Magnetic, microplate-format plasmid isolation protocol for high-yield, sequencing-grade DNA. Biotechniques October 2000;29(4):786-8, 790, 792

Konecki D S, Phillips J J. TurboPrep II: an inexpensive, high-throughput plasmid template preparation protocol. Biotechniques February 1998;24(2):286-8, 290-3

Garner H R, Amstrong B, Kramarsky D A. High-throughput DNA preparation system. Genet Anal Tech Appl October-December 1992;9(5-6):134-9

Huang G M, Wang K, Kuo C, Paeper B, Hood L. A high-throughput plasmid DNA preparation method. Anal Biochem 1994 Nov. 15;223(1):35-8

Beg O U, Holt R G. A cost-effective plasmid purification protocol suitable for fluorescent automated DNA sequencing. Mol Biotechnol February 1998;9(1):79-83

Pogue R R, Cook M E, Livingstone L R, Hunt S W 3rd. Preparation of template for automated sequencing using QIAGEN resin. Biotechniques September 1993;15(3):376-8, 381

Kirschner L S, Stratakis C A. Large-scale preparation of sequence-ready bacterial artificial chromosome DNA using QIAGEN columns. Biotechniques July 1992;27(1):72-4

We claim:

1. A method for isolating RNA, DNA or protein from an animal tissue sample comprising the steps of:
beadmilling the tissue sample in a beadmilling buffer to disrupt cells in the sample; and
extracting RNA, DNA or protein from the tissue sample using a solid phase extraction method.

2. A method for isolating RNA from an animal tissue sample comprising the steps of:
preserving the tissue sample in a RNA preservation buffer;
switching the tissue sample to a beadmilling buffer that contains a ribonuclease inhibitor;
beadmilling the tissue sample in the beadmilling buffer to disrupt cells in the sample; and
extracting RNA from the tissue sample using a solid phase extraction method.

3. The method of claim 2, wherein the RNA preservation buffer is RNAlater™.

4. The method of claim 2, wherein the tissue is beadmilled with glass beads.

5. The method of claim 2, wherein the beadmilling step also shears DNA released from the disrupted cells.

6. The method of claim 2, wherein the ribonuclease inhibitor in the beadmilling buffer is guanidium isothiocyanate.

7. The method of claim 4, wherein the glass beads have a diameter of about 2 millimeters to about 5 millimeters.

8. The method of claim 4, wherein the glass beads have a diameter of about 3 millimeters to about 4 millimeters.

9. The method of claim 4, wherein the glass beads have a diameter of about 3 millimeters.

10. The method of claim 7, wherein the volume ratio of the beadmilling buffer to the glass beads is from about 1.3 to 1, to about 79.6 to 1.

11. The method of claim 7, wherein the volume ratio of the beadmilling buffer to the glass beads is from about 2.6 to 1, to about 39.8 to 1.

12. The method of claim 7, wherein the volume ratio of the beadmilling buffer to the glass beads is about 11.8 to 1.

13. The method of claim 4, wherein the beadmilling step includes 5 to 20 shaking cycles.

14. The method of claim 13, wherein each shaking cycle lasts from about 30 seconds to about 60 seconds.

15. The method of claim 13, wherein each shaking cycle lasts about 45 seconds.

16. The method of claim 13, further comprising the step of cooling the tissue sample to a temperature between 0° C. and 25° C. every 1 to 5 shaking cycles.

17. The method of claim 16, wherein the tissue sample is cooled to about 4° C..

18. The method of claim 4, wherein the beadmilling step includes 6 to 9 shaking cycles.

19. The method of claim 4, wherein the beadmilling step includes 14 or 15 shaking cycles.

20. The method of claim 4, wherein beadmilling constitutes shaking in the presence of glass beads at a speed from about 3 meters per second to about 10 meters per second.

21. The method of claim 4, wherein beadmilling constitutes shaking in the presence of glass beads at a speed from about 5 meters per second to about 8 meters per second.

22. The method of claim 4, wherein beadmilling constitutes shaking in the presence of glass beads at a speed of about 6.5 meters per second.
